(19) United States
(12) Patent Application Publication
(10) Pub. No.: US 2025/0364109 A1
(43) Pub. Date: Nov. 27, 2025

(54) Scanning Procedure Determination

(71) Applicant: Siemens Healthineers AG, Forchheim (DE)

(72) Inventors: Zhi Gen Yang, Shenzhen (CN); Zhi Cheng Deng, Shenzhen (CN); Wei Lin Wang, Shenzhen (CN); Dai Cheng Xie, Shenzhen (CN)

(73) Assignee: Siemens Healthineers AG, Forchheim (DE)

(21) Appl. No.: 19/215,451

(22) Filed: May 22, 2025

(30) Foreign Application Priority Data
May 24, 2024 (CN) 202410662184.1

(51) Int. Cl. G16H 20/40

(52) U.S. Cl. CPC G16H 20/40

(57) ABSTRACT

A method and apparatus for determining a scanning procedure, including: pre-acquiring historical scanning data including scanning request information and scanning procedures, and establishing a scanning procedure inference model based on the historical scanning data; receiving scanning request information of a current patient, and inferring using the scanning procedure inference model based on the scanning request information to obtain a scanning procedure recommendation list including different recommendation probabilities; and providing the scanning procedure recommendation list to a user for selection.

### TECHNICAL FIELD

The present disclosure relates to the technical field of Magnetic Resonance (MR) imaging, and in particular to a method and apparatus for determining a scanning procedure, a system, a computer-readable storage medium, and a computer program product.

### BACKGROUND

A Magnetic Resonance Imaging (MRI) system is based on a magnetic field generated by a magnet and uses the nuclear magnetic resonance phenomenon to obtain information on the molecular structure and internal structure of a human body, etc.

Before performing an MRI scan, a technician must complete the registration of a patient. On the one hand, the technician needs to check the patient's basic information, which usually comes from a Radiology Information System (RIS); on the other hand, the technician needs to select an appropriate scanning procedure (also referred to as a protocol set) and a body part to perform the current scan.

For scanning procedure selection, the technician needs to open a procedure selection tab, select a protocol “tree map”, select an “area”, then select an “examination”, and finally select a “procedure”. It substantially needs 4-6 click selection operations to select a final procedure. Not only is the selection process time-consuming, but it also requires the technician to make selections based on their own experience. Especially when the patient is registered in a mobile device (e.g., a touch tablet), it will take more time to find a procedure and select it. In the absence of an RIS, the technician needs to register locally at a scanning site and requires the above process to select an appropriate procedure.

To this end, those skilled in the art are still working toward seeking other solutions for determining the scanning procedure.

### SUMMARY

In view of this, aspects of the present application propose a method and apparatus for determining a scanning procedure, a system, a medium, and a program product to improve the efficiency of selecting a scanning procedure.

A method for determining a scanning procedure proposed in an aspect of the present application comprises: pre-acquiring historical scanning data including scanning request information and scanning procedures, and establishing a scanning procedure inference model on the basis of the historical scanning data; receiving scanning request information of a current patient, and inferring by means of the scanning procedure inference model on the basis of the scanning request information to obtain a scanning procedure recommendation list including different recommendation probabilities; and providing the scanning procedure recommendation list to a user for selection.

In an implementation, while providing the scanning procedure recommendation list to the user for selection, the method further comprises: pre-filling the scanning procedure with the highest recommendation probability in the scanning procedure recommendation list into a scanning procedure option of the patient's scan plan.

In an implementation, the scanning request information includes scanning area information and other related information; and establishing the scanning procedure inference model on the basis of the historical scanning data comprises: storing the historical scanning data in a structured manner, and establishing, on the basis of the structured and stored historical scanning data, a scanning procedure inference model based on key field search; wherein key fields in the structured and stored historical scanning data, including scanning areas, other related information and scanning procedures, have been associated with standardized field descriptions on the basis of semantic analysis.

In an implementation, inferring by means of the scanning procedure inference model on the basis of the scanning request information to obtain the scanning procedure recommendation list including different recommendation probabilities comprises: extracting scanning area information from the scanning request information, and determining an area to be scanned of the patient according to the scanning area information; and, using a standardized field description corresponding to the area to be scanned as a first key field, and using at least one kind of other related information in the scanning request information as an auxiliary key field, performing matching retrieval from the structured and stored historical scanning data by means of the scanning procedure inference model, and determining a corresponding recommendation probability according to a corresponding degree of matching, so as to obtain the scanning procedure recommendation list including different recommendation probabilities.

In an implementation, the method further comprises: acquiring a selection preference set by the user, and using the selection preference as a second key field; wherein the selection preference includes any one of standardization, speed focus, motion insensitivity and full automation; and performing matching retrieval from the structured and stored historical scanning data is: performing matching retrieval from the structured and stored historical scanning data on the basis of the first key field, the auxiliary key field, and the second key field.

In an implementation, storing the historical scanning data in the structured manner comprises: storing the historical scanning data in a database on the basis of the standardized field descriptions; or storing the historical scanning data in a knowledge graph, wherein the knowledge graph includes standardized field description nodes, key field nodes in the historical scanning data, and multiple edges representing a relationship between the nodes; and the relationship between the nodes includes: a relationship between the standardized field description nodes and the key field nodes in the historical scanning data, and a relationship between different key nodes in same historical scanning data.

In an implementation, establishing the scanning procedure inference model on the basis of the historical scanning data comprises: using scanning request information in each piece of historical scanning data as an input sample, using a scanning procedure in the historical scanning data as an output sample to train a convolutional neural network, and obtaining a trained scanning procedure inference model; and inferring by means of the scanning procedure inference model on the basis of the scanning request information to obtain the scanning procedure recommendation list including different recommendation probabilities comprises: using the scanning request information as an input of the scanning procedure inference model, and obtaining the scanning procedure recommendation list including different recommendation probabilities output by the scanning procedure inference model.

In an implementation, the method further comprises: acquiring a selection preference set by the user, and using the selection preference as a key field; wherein the selection preference includes any one of standardization, speed focus, motion insensitivity and full automation; and inferring by means of the scanning procedure inference model on the basis of the scanning request information to obtain the scanning procedure recommendation list including different recommendation probabilities further comprises: filtering, on the basis of the key field, the scanning procedure recommendation list including different recommendation probabilities output by the scanning procedure inference model to obtain a scanning procedure recommendation list that meets the selection preference.

In an implementation, the method further comprises: receiving a scanning procedure currently determined by the user; and updating the scanning procedure inference model by using the information of the current patient, including the scanning request information and the scanning procedure as new historical scanning data.

An apparatus for determining a scanning procedure proposed in an aspect of the present application comprises: a first module configured to pre-acquire historical scanning data including scanning request information and scanning procedures, and to establish a scanning procedure inference model on the basis of the historical scanning data; a second module configured to receive scanning request information of a current patient, and to infer by means of the scanning procedure inference model on the basis of the scanning request information to obtain a scanning procedure recommendation list including different recommendation probabilities; and a third module configured to provide the scanning procedure recommendation list to a user for selection.

In an implementation, while providing the scanning procedure recommendation list to the user for selection, the third module is further configured to pre-fill the scanning procedure with the highest recommendation probability in the scanning procedure recommendation list into a scanning procedure option of the patient's scan plan.

In an implementation, the scanning request information includes scanning area information and other related information; and the first module stores the historical scanning data in a structured manner, and establishes, on the basis of the structured and stored historical scanning data, a scanning procedure inference model based on key field search; wherein key fields in the structured and stored historical scanning data, including scanning areas, other related information and scanning procedures, have been associated with standardized field descriptions on the basis of semantic analysis.

In an implementation, the second module determines an area to be scanned of the patient according to the scanning area information; and, using a standardized field description corresponding to the area to be scanned as a first key field, and using at least one kind of other related information in the scanning request information as an auxiliary key field, performs matching retrieval from the structured and stored historical scanning data by means of the scanning procedure inference model, and determines a corresponding recommendation probability according to a corresponding degree of matching, so as to obtain the scanning procedure recommendation list including different recommendation probabilities.

In an implementation, the second module further acquires a selection preference set by the user, uses the selection preference as a second key field, and performs matching retrieval from the structured and stored historical scanning data on the basis of the first key field, the auxiliary key field, and the second key field; wherein the selection preference includes any one of standardization, speed focus, motion insensitivity and full automation.

In an implementation, the first module uses scanning request information in each piece of historical scanning data as an input sample, uses a scanning procedure in the historical scanning data as an output sample to train a convolutional neural network, and obtains a trained scanning procedure inference model; and the second module uses the scanning request information as an input of the scanning procedure inference model, and obtains the scanning procedure recommendation list including different recommendation probabilities output by the scanning procedure inference model.

Another apparatus for determining a scanning procedure proposed in an aspect of the present application comprises: at least one memory storing a computer program; and at least one processor configured to read and execute the computer program to implement the method for determining the scanning procedure as described above.

An MRI system proposed in an aspect of the present application comprises: the apparatus for determining the scanning procedure in any one of the implementations described above.

A computer program product proposed in an aspect of the present application comprises a computer program; wherein, when the computer program is executed by a processor, the method for determining the scanning procedure as described above is implemented.

It can be seen from the above solutions that in the technical solutions in the aspects of the present application, since the historical scanning data including the patient's scanning request information and the scanning procedure are pre-acquired, and the scanning procedure inference model is established on the basis of the historical scanning data, after receiving the scanning request information of the current patient, the scanning procedure recommendation list including different recommendation probabilities can be inferred by means of the scanning procedure inference model on the basis of the scanning request information, and the scanning procedure recommendation list is provided to the user for selection, thereby accelerating the selection of the scanning procedure. Further, the scanning procedure with the highest recommendation probability in the scanning procedure recommendation list may also be pre-filled into the scanning procedure option of the patient's scan plan for the user to determine and select, thereby further accelerating the selection of the scanning procedure. It can be seen that the user does not need to make multiple selections from the tree-like scanning procedure options layer by layer, which improves the efficiency of determining the scanning procedure.

### BRIEF DESCRIPTION OF THE DRAWINGS

Preferred aspects of the present application are described in detail below with reference to the drawings, to give those skilled in the art a clearer understanding of the above and other features and advantages of the present application. In the drawings:

FIG. 1 is an exemplary flowchart of a method for determining a scanning procedure in an aspect of the present application.

FIG. 2 is an exemplary structural diagram of an apparatus for determining a scanning procedure in an aspect of the present application.

FIG. 3 is an exemplary structural diagram of another apparatus for determining a scanning procedure in an aspect of the present application.

In the figures, the reference numerals are as follows:

| Reference numeral | Meaning |
|---|---|
| 101, 102, 103 | Steps |
| 201 | First module |
| 202 | Second module |
| 203 | Third module |
| 204 | Fourth module |
| 301 | Memory |
| 302 | Processor |
| 303 | Bus |

### DETAILED DESCRIPTION

In aspects of the present application, in order to improve the efficiency of selecting a scanning procedure, it is considered to provide an intelligent recommendation mechanism.

To enable a clearer understanding of the objective, technical solutions, and effects of the present application, particular aspects of the present application are now explained with reference to the accompanying drawings, in which identical labels indicate structurally identical components or components with similar structures but identical functions.

As used herein, “exemplary” and “schematic” mean “serving as an instance, example, or illustration”. No drawing or aspect described herein as “exemplary” or “schematic” should be interpreted as a more preferred or more advantageous technical solution.

To make the drawings appear uncluttered, only those parts relevant to the present application are shown schematically in the drawings; they do not represent the actual structure thereof as a product.

In this text, “a” does not only mean “just this one”; it may also mean “more than one”. In this text, “first”, “second”, etc., are merely used to differentiate between parts, not to indicate the order or degree of importance between parts, etc.

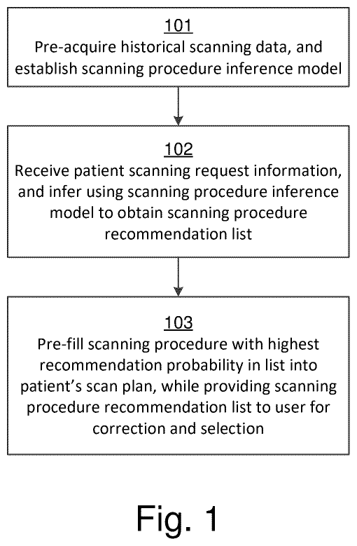

FIG. 1 is an exemplary flowchart of a method for determining a scanning procedure in an aspect of the present application. As shown in FIG. 1, the method may comprise the following processing:

Step 101: Pre-acquire historical scanning data, including scanning request information and scanning procedures, and establish a scanning procedure inference model on the basis of the historical scanning data. The scanning request information may include scanning area information and other related information.

In this step, when a particular implementation is performed, multiple implementation methods are possible. Two of them are listed below:

First method: The historical scanning data is stored in a structured manner, and a scanning procedure inference model based on key field search is established on the basis of the structured and stored historical scanning data; wherein key fields in the structured and stored historical scanning data, including scanning areas, other related information and scanning procedures, have been associated with standardized field descriptions on the basis of semantic analysis.

Storing the historical scanning data in the structured manner may comprise: storing the historical scanning data in a database on the basis of the standardized field descriptions. Alternatively, it may comprise: storing the historical scanning data in a knowledge graph, the knowledge graph including standardized field description nodes, key field nodes in the historical scanning data, and multiple edges representing the relationship between the nodes. The relationship between the nodes includes: the relationship between the standardized field description nodes and the key field nodes in the historical scanning data, and the relationship between different key nodes in the same historical scanning data.

Second method: Scanning request information in each piece of historical scanning data is used as an input sample, a scanning procedure in the historical scanning data is used as an output sample to train a convolutional neural network, and a trained scanning procedure inference model is obtained.

Step 102: Receive scanning request information of a current patient, and infer by means of the scanning procedure inference model on the basis of the scanning request information to obtain a scanning procedure recommendation list including different recommendation probabilities.

The scanning request information of the current patient may be obtained from RIS or pre-registration results. In an implementation, the information of the current patient may include an admitting diagnosis description, a request procedure, alert information, a physician, and other information. The request procedure usually provides information related to a scanning area, so scanning area information can be obtained from the request procedure. The admitting diagnosis description, the alert information, the physician, and other information are other related information. Of course, due to the differences in RIS systems of different hospitals, the scanning area information may also be stored in other fields of the scanning request information, such as a review field, an attachment field, etc., but the scanning area information may always be obtained from the corresponding fields. For example, the scanning request information of some patients is listed in Table 1 below:

TABLE 1

| | | Admitting Diagnosis | | |
|---|---|---|---|---|
| | Request Procedure | Description | Alert | Physician |
| 1 | MRI brain without | Admission diagnosis: | Gadolinium | ZHANG San |
| | contrast | headache | allergy | |
| 2 | MRI heart scan | Admission diagnosis: | Claustrophobia | LI Si |
| | | myocarditis | | |
| 3 | MRI tumor liver | Admission diagnosis: | Claustrophobia | WANG Wu |
| | scan | cancer risk | | |

In this step, in an implementation corresponding to the first implementation method in step 101, scanning area information may be extracted from the scanning request information, and an area to be scanned of the patient may be determined according to the scanning area information. The standardized field description corresponding to the area to be scanned is used as a first key field, and at least one kind of other related information in the scanning request information is used as an auxiliary key field, for example the admitting diagnosis description and/or the alert are used as the auxiliary key field. Matching retrieval is performed by means of the scanning procedure inference model from the structured and stored historical scanning data, and a corresponding recommendation probability is determined according to a corresponding degree of matching, so as to obtain a scanning procedure recommendation list including different recommendation probabilities, for example a scanning procedure recommendation list arranged from high to low or from low to high according to the recommendation probabilities.

Further, a user may also set a selection preference. For example, in an implementation, the selection preference includes any one of standardization, speed focus, motion insensitivity, and full automation. Correspondingly, in this step, the selection preference set by the user may be acquired and used as a second key field. Then, matching retrieval is performed by means of the scanning procedure inference model from the structured and stored historical scanning data on the basis of the first key field, the auxiliary key field, and the second key field.

Corresponding to the second implementation method in step 101, in this step, the scanning request information is used as an input of the scanning procedure inference model, and the scanning procedure recommendation list including different recommendation probabilities output by the scanning procedure inference model is obtained.

Similarly, when the user sets a selection preference, the selection preference set by the user may be acquired and used as a key field. Then, on the basis of the key field, the scanning procedure recommendation list, including different recommendation probabilities output by the scanning procedure inference model, is filtered to obtain a scanning procedure recommendation list that meets the selection preference.

Step 103: Pre-fill the scanning procedure with the highest recommendation probability in the scanning procedure recommendation list into a scanning procedure option of the patient's scan plan for the user to determine and select, while providing the scanning procedure recommendation list to the user for correction and selection. Of course, in other implementations, the scanning procedure with the highest recommendation probability in the scanning procedure recommendation list may not be pre-filled into the scanning procedure option of the patient's scan plan, but only the scanning procedure recommendation list is provided to the user, and the user directly selects from the scanning procedure recommendation list.

For example, with respect to the scanning request information of some patients in Table 1, it is assumed that the recommended procedure list provided for each patient is as shown in Table 2 below.

TABLE 2

| | | Admitting | | |
|---|---|---|---|---|
| | Request | Diagnosis | | |
| | Procedure | Description | *** | Recommended Procedure List |
| 1 | MRI brain | Admission | *** | Brain assisted - speed priority with a |
| | without | diagnosis: | | probability of 90% |
| | contrast | headache | | Brain full automation with a probability of |
| | | | | 50% |
| 2 | MRI heart | Admission | *** | Heart assisted - myocarditis with a probability |
| | scan | diagnosis: | | of 95% |
| | | myocarditis | | Heart assisted - standard with a probability of |
| | | | | 75% |
| 3 | MRI tumor | Admission | *** | Abdominal assisted - bolus tracking - motion |
| | liver scan | diagnosis: | | sensitivity with a probability of 90% |
| | | cancer risk | | Abdominal assisted - bolus tracking - breath |
| | | | | holding with a probability of 60% |

After the user determines the scanning procedure to be adopted for the patient, the patient may be scanned on the basis of the scanning procedure to obtain an MRI.

Meanwhile, after receiving the scanning procedure currently determined by the user, it is also possible to update the scanning procedure inference model by using the information of the current patient, including the scanning request information, the scanning area information, and the scanning procedure as new historical scanning data.

The method for determining the scanning procedure in the aspect of the present application has been described in detail above, and an apparatus for determining a scanning procedure in an aspect of the present application will be described in detail below. The apparatus for determining the scanning procedure in the aspect of the present application may be used to implement the method for determining the scanning procedure in the aspect of the present application. For details not disclosed in the apparatus aspect of the present application, reference can be made to the corresponding description in the method aspect of the present application, and the details will not be described one by one again here.

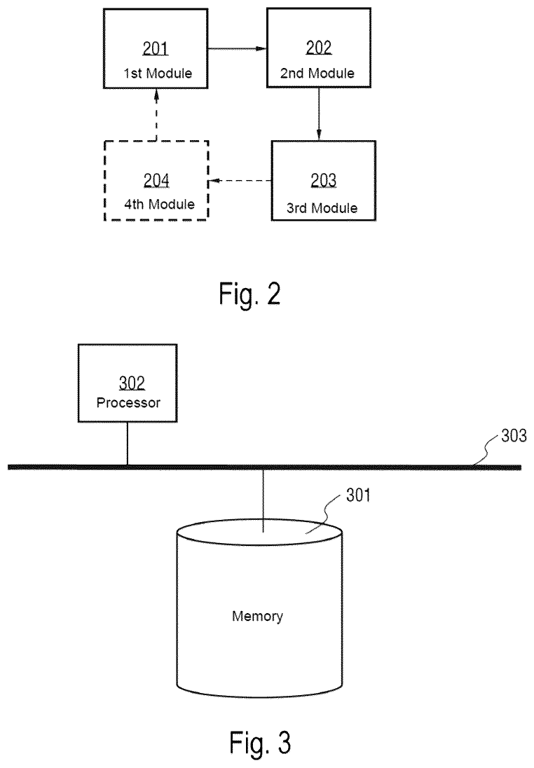

FIG. 2 is an exemplary structural diagram of an apparatus for determining a scanning procedure in an aspect of the present application. As shown in the solid line part in FIG. 2, the apparatus may comprise a first module 201, a second module 202, and a third module 203.

The first module 201 is configured to pre-acquire historical scanning data,

including scanning request information and scanning procedures, and to establish a scanning procedure inference model on the basis of the historical scanning data.

The second module 202 is configured to receive scanning request information of a current patient, and to infer by means of the scanning procedure inference model on the basis of the scanning request information to obtain a scanning procedure recommendation list including different recommendation probabilities.

The third module 203 is configured to pre-fill the scanning procedure with the highest recommendation probability in the scanning procedure recommendation list into a scanning procedure option of the patient's scan plan for the user to determine and select while providing the scanning procedure recommendation list to the user for correction and selection. Of course, in other implementations, the third module 203 may not pre-fill the scanning procedure with the highest recommendation probability in the scanning procedure recommendation list into the scanning procedure option of the patient's scan plan, but only provides the scanning procedure recommendation list to the user, and the user directly selects from the scanning procedure recommendation list.

In an implementation, the first module 201 may store the pre-acquired historical scanning data in a structured manner, and establish, on the basis of the structured and stored historical scanning data, a scanning procedure inference model based on key field search; wherein key fields in the structured and stored historical scanning data, including scanning areas, other related information and scanning procedures, have been associated with standardized field descriptions on the basis of semantic analysis. Storing the historical scanning data in the structured manner may comprise: storing the historical scanning data in a database on the basis of the standardized field descriptions. Alternatively, it may comprise: storing the historical scanning data in a knowledge graph, the knowledge graph including standardized field description nodes, key field nodes in the historical scanning data, and multiple edges representing the relationship between the nodes. The relationship between the nodes includes: the relationship between the standardized field description nodes and the key field nodes in the historical scanning data, and the relationship between different key nodes in the same historical scanning data.

Correspondingly, the second module 202 may determine an area to be scanned of the patient according to the scanning area information extracted from the information of the current patient; and use a standardized field description corresponding to the area to be scanned as a first key field, use at least one kind of other related information in the scanning request information as an auxiliary key field, perform matching retrieval from the structured and stored historical scanning data by means of the scanning procedure inference model, and determine a corresponding recommendation probability according to a corresponding degree of matching, so as to obtain the scanning procedure recommendation list including different recommendation probabilities.

In addition, the second module 202 may further acquire a selection preference set by the user, use the selection preference as a second key field, and perform matching retrieval from the structured and stored historical scanning data on the basis of the first key field, the auxiliary key field, and the second key field; wherein the selection preference includes any one of standardization, speed focus, motion insensitivity and full automation.

In another implementation, the first module 201 may use scanning request information in each piece of historical scanning data as an input sample, use a scanning procedure in the historical scanning data as an output sample to train a convolutional neural network, and obtain a trained scanning procedure inference model.

Correspondingly, the second module 202 may use the scanning request information of the current patient as an input of the scanning procedure inference model, and obtain the scanning procedure recommendation list including different recommendation probabilities output by the scanning procedure inference model.

In addition, the second module 202 may further acquire a selection preference set by the user and use the selection preference as a key field, and then, on the basis of the key field, filter the scanning procedure recommendation list including different recommendation probabilities output by the scanning procedure inference model to obtain a scanning procedure recommendation list that meets the selection preference.

In addition, the apparatus may further comprise, as shown in the dotted part of FIG. 2: a fourth module 204 configured to receive a scanning procedure currently determined by the user; and to provide the information of the current patient including the scanning request information and the scanning procedure as new historical scanning data to the first module 201, so that the first module 201 updates the scanning procedure inference model on the basis of the new historical scanning data.

In practice, the apparatus for determining the scanning procedure provided in this implementation of the present application can be specifically implemented in various ways. For example, an eco-design assessment apparatus may be compiled into a plug-in installed in a smart terminal by using an application programming interface that complies with specific rules, or may be packaged into an application program to constitute a program product for users to download and use.

When compiled as a plug-in, the apparatus for determining the scanning procedure may be implemented in a variety of plug-in forms, such as ocx, dll and cab. The apparatus for determining the scanning procedure provided in this implementation of the present application may also be implemented by using specific technologies, such as Flash plug-in technology, RealPlayer plug-in technology, MMS plug-in technology, MIDI plug-in technology, or ActiveX plug-in technology.

The method for determining the scanning procedure provided in this implementation of the present application may be packaged into an application program in the form of instructions or instruction sets to form a program product for users to download and use, or be stored in various storage media in the form of instructions or instruction sets. These storage media comprise, but are not limited to: floppy disks, optical disks, DVDs, hard disks, flash memory, USB flash memory, CF cards, SD cards, SDHC cards, MMC cards, SM cards, memory sticks, and xD cards.

It should be apparent that an operating system operating in a computer may implement the functions of any one of the above aspects not only by executing program codes read by the computer from a storage medium, but also by using instructions based on the program codes to implement part or all of the actual operations.

For example, FIG. 3 is an exemplary structural diagram of another apparatus for determining a scanning procedure in an aspect of the present application. The apparatus may be configured to execute the method shown in FIG. 1, or to implement the apparatus in FIG. 2. As shown in FIG. 3, the apparatus may comprise at least one memory 301 and at least one processor 302. In addition, the apparatus may further comprise some other components, such as a communication port, an input/output controller, a network communication interface, etc. These components communicate via a bus 303, etc.

The at least one memory 301 is configured to store a computer program. In an example, the computer program may be understood to comprise various modules of the apparatus shown in FIG. 2. Additionally, the at least one memory 301 may store an operating system, etc. The operating system comprises, but is not limited to: an Android operating system, a Symbian operating system, a Windows operating system, a Linux operating system, etc.

The at least one processor 302 is configured to call up the computer program stored in the at least one memory 301, in order to perform the method for determining the scanning procedure described in the aspect of the present application. The first processor 302 may be a CPU, a processing unit/module, an ASIC, a logic module, a programmable gate array, or the like, and may receive and send data through a communication port.

It should be understood that “and/or” used herein is intended to include any and all possible combinations of one or more of associated listed items.

The number of aspects of the present application is only used for description and does not represent the advantages of the aspects.

In the technical solutions in the aspects of the present application, since the historical scanning data including the patient's scanning request information and the scanning procedure are pre-acquired, and the scanning procedure inference model is established on the basis of the historical scanning data, after receiving the scanning request information of the current patient, the scanning procedure recommendation list including different recommendation probabilities can be inferred by means of the scanning procedure inference model on the basis of the scanning request information, and the scanning procedure recommendation list is provided to the user for selection, thereby accelerating the selection of the scanning procedure. Further, the scanning procedure with the highest recommendation probability in the scanning procedure recommendation list may also be pre-filled into the scanning procedure option of the patient's scan plan for the user to determine and select, thereby further accelerating the selection of the scanning procedure. It can be seen that the user does not need to make multiple selections from the tree-like scanning procedure options layer by layer, which improves the efficiency of determining the scanning procedure.

The aspects above are merely preferred aspects of the present application, which are not intended to limit it. Any amendments, equivalent substitutions, or improvements, etc., made within the spirit and principles of the present application shall be included in the scope of protection thereof.

1. A method for determining a scanning procedure of a magnetic resonance imaging system, comprising:
pre-acquiring historical scanning data, including scanning request information and scanning procedures, and establishing a scanning procedure inference model based on the historical scanning data;
receiving scanning request information of a current patient, and inferring using the scanning procedure inference model based on the scanning request information to obtain a scanning procedure recommendation list, including different recommendation probabilities; and
providing the scanning procedure recommendation list to a user for selection.

2. The method according to claim 1, wherein while providing the scanning procedure recommendation list to the user for selection, the method further comprises: pre-filling the scanning procedure with a highest recommendation probability in the scanning procedure recommendation list into a scanning procedure option of a patient's scan plan.

3. The method according to claim 1,
wherein the scanning request information includes scanning area information and other related information; and
wherein establishing the scanning procedure inference model based on the historical scanning data comprises:
storing the historical scanning data in a structured manner; and
establishing, based on the structured and stored historical scanning data, a scanning procedure inference model based on key field search, wherein key fields in the structured and stored historical scanning data, including scanning areas, other related information, and scanning procedures, have been associated with standardized field descriptions based on semantic analysis.

4. The method according to claim 3, wherein inferring using the scanning procedure inference model based on the scanning request information to obtain the scanning procedure recommendation list including different recommendation probabilities comprises:
extracting scanning area information from the scanning request information, and determining an area to be scanned of the patient according to the scanning area information;
and, using a standardized field description corresponding to the area to be scanned as a first key field, and using at least one kind of other related information in the scanning request information as an auxiliary key field, performing matching retrieval from the structured and stored historical scanning data using the scanning procedure inference model; and
determining a corresponding recommendation probability according to a corresponding degree of matching, so as to obtain the scanning procedure recommendation list including different recommendation probabilities.

5. The method according to claim 4, wherein the method further comprises:
acquiring a selection preference set by the user, and using the selection preference as a second key field, wherein the selection preference includes any one of standardization, speed focus, motion insensitivity, and full automation; and
performing matching retrieval from the structured and stored historical scanning data is based on the first key field, the auxiliary key field, and the second key field.

6. The method according to claim 3, wherein storing the historical scanning data in the structured manner comprises: storing the historical scanning data in a database based on the standardized field descriptions; or storing the historical scanning data in a knowledge graph, wherein the knowledge graph includes standardized field description nodes, key field nodes in the historical scanning data, and multiple edges representing a relationship between the nodes; and the relationship between the nodes includes: a relationship between the standardized field description nodes and the key field nodes in the historical scanning data, and a relationship between different key nodes in same historical scanning data.

7. The method according to claim 1, wherein establishing the scanning procedure inference model based on the historical scanning data comprises: using scanning request information in each piece of historical scanning data as an input sample, using a scanning procedure in the historical scanning data as an output sample to train a convolutional neural network, and obtaining a trained scanning procedure inference model; and
inferring using the scanning procedure inference model based on the scanning request information to obtain the scanning procedure recommendation list including different recommendation probabilities comprises: using the scanning request information as an input of the scanning procedure inference model, and obtaining the scanning procedure recommendation list including different recommendation probabilities output by the scanning procedure inference model.

8. The method according to claim 7, wherein the method further comprises: acquiring a selection preference set by the user, and using the selection preference as a key field, wherein the selection preference includes any one of standardization, speed focus, motion insensitivity, and full automation; and
inferring using the scanning procedure inference model based on the scanning request information to obtain the scanning procedure recommendation list including different recommendation probabilities further comprises: filtering, based on the key field, the scanning procedure recommendation list including different recommendation probabilities output by the scanning procedure inference model to obtain a scanning procedure recommendation list that meets the selection preference.

9. The method according to claim 1, wherein the method further comprises:
receiving a scanning procedure currently determined by the user; and
updating the scanning procedure inference model by using the information of the current patient including the scanning request information and the scanning procedure as new historical scanning data.

10. An apparatus for determining a scanning procedure of a magnetic resonance imaging system, comprising:
a first module configured to pre-acquire historical scanning data including scanning request information and scanning procedures, and to establish a scanning procedure inference model based on the historical scanning data;
a second module configured to receive scanning request information of a current patient, and to infer using the scanning procedure inference model based on the scanning request information to obtain a scanning procedure recommendation list including different recommendation probabilities; and
a third module configured to provide the scanning procedure recommendation list to a user for selection.

11. The apparatus according to claim 10, wherein while providing the scanning procedure recommendation list to the user for selection, the third module is further configured to pre-fill the scanning procedure with a highest recommendation probability in the scanning procedure recommendation list into a scanning procedure option of a patient's scan plan.

12. The apparatus according to claim 10, wherein the scanning request information includes scanning area information and other related information, and the first module is configured to store the historical scanning data in a structured manner, and establish, based on the structured and stored historical scanning data, a scanning procedure inference model based on key field search, wherein key fields in the structured and stored historical scanning data, including scanning areas, other related information and scanning procedures, have been associated with standardized field descriptions based on semantic analysis.

13. The apparatus according to claim 12, wherein the second module is configured to determine an area to be scanned of the patient according to the scanning area information; and, using a standardized field description corresponding to the area to be scanned as a first key field, and using at least one kind of other related information in the scanning request information as an auxiliary key field, perform matching retrieval from the structured and stored historical scanning data using the scanning procedure inference model, and determine a corresponding recommendation probability according to a corresponding degree of matching, so as to obtain the scanning procedure recommendation list including different recommendation probabilities.

14. The apparatus according to claim 13, wherein the second module is further configured to acquire a selection preference set by the user, use the selection preference as a second key field, and perform matching retrieval from the structured and stored historical scanning data based on the first key field, the auxiliary key field, and the second key field; wherein the selection preference includes any one of standardization, speed focus, motion insensitivity and full automation.

15. The apparatus according to claim 10, wherein the first module is configured to use scanning request information in each piece of historical scanning data as an input sample, use a scanning procedure in the historical scanning data as an output sample to train a convolutional neural network, and obtain a trained scanning procedure inference model; and
the second module is configured to use the scanning request information as an input of the scanning procedure inference model, and obtain the scanning procedure recommendation list including different recommendation probabilities output by the scanning procedure inference model.

16. An apparatus for determining a scanning procedure of a magnetic resonance imaging system, comprising:
at least one memory storing a computer program; and
at least one processor configured to read and execute the computer program to implement the method for determining the scanning procedure according to claim 1.

17. A magnetic resonance imaging system, comprising the apparatus for determining the scanning procedure of the magnetic resonance imaging system according to claim 10.

18. A non-transitory computer program product, comprising a computer program, wherein when the computer program is executed by a processor, the method for determining the scanning procedure of the magnetic resonance imaging system according to claim 1 is implemented.
